(19) United States
(12) Patent Application Publication
(10) Pub. No.: US 2024/0082557 A1
(43) Pub. Date: Mar. 14, 2024

(54) LARGE DIAMETER HEMOSTASIS VALVES

(71) Applicant: NEPTUNE MEDICAL INC., Burlingame, CA (US)

(72) Inventors: Alexander Q. TILSON, Burlingame, CA (US); Garrett J. GOMES, Pleasant Hill, CA (US); Charles Steele LOVE, Santa Barbara, CA (US); Eugene F. DUVAL, Menlo Park, CA (US)

(21) Appl. No.: 18/262,904

(22) PCT Filed: Jan. 25, 2022
(86) PCT No.: PCT/US2022/013636

§ 371 Date: Jul. 25, 2023

Related U.S. Application Data
(60) Provisional application No. 63/141,392, filed on Jan. 25, 2021.

(51) Int. Cl. A61M 39/06

(52) U.S. Cl. CPC A61M 39/0613

(57) ABSTRACT

A hemostasis valve includes a housing having a central bore therethrough, a compression tube positioned within the central bore, a soft annular elastomeric seal positioned within the central bore distal to the compression tube, and at least one actuator coupled to the housing. Movement of the actuator relative to the housing may move the compression tube towards or away from the annular elastomeric seal to increase adjust the inner diameter of the annular elastic seal. The large-bore hemostatic valves described herein may be particularly well suited for use with controllably adjusting the seal pressure around a medical device (e.g., catheter) inserted through the valve and may maintain a tight seal at high pressure and with large diameter medical devices.

### CLAIM OF PRIORITY

This patent application claims priority to U.S. provisional patent application No. 63/141,392, titled “LARGE DIAMETER HEMOSTASIS VALVES,” and filed on Jan. 25, 2021, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated.

### BACKGROUND

As catheters and introducers enter the vasculature, they create a port through which pressurized blood (venous or arterial) can exit and leave the body. This blood loss is detrimental to the patient's health and creates a messy and dangerous working environment. Therefore, many of these devices have a hemostasis valve at the proximal end to prevent blood loss. Currently available hemostasis valves, however, are designed primarily for use with small bore devices. Additionally, many current hemostasis valves leak, can be pushed out of the body at high hemostatic pressures, cause drag along devices extended therethrough, can be difficult or cumbersome to operate, and/or cannot be adjusted to work with devices of varying diameters. Accordingly, a large bore hemostasis valve that solves some or all of these problems is desired.

### SUMMARY OF THE DISCLOSURE

In general, described herein are hemostatic valve apparatuses (e.g., devices, systems, etc.) that may be configured as large-bore hemostatic valves capable of withstanding high pressures with minimal or no leaking. The apparatuses may include an extremely soft (e.g., low Shore 00 durometer) annular elastomeric seal within a central bore of a housing. The annular elastomeric seal may be arranged adjacent to a compression tube that is axially and/or rotationally slidable within the central bore to compress and seal the central opening through the annulus of the annular elastomeric seal. The compression tube may be coupled to (e.g., either directly or indirectly bonded to) the annular elastomeric seal. The axial and/or rotational position of the compression tube within the central bore may be controlled by one or more actuators that are movably coupled to the housing. The actuator may have one or more engagement surfaces engaging with one or more driving surfaces on or coupled to the compression tube.

For example, a hemostasis valve may include a housing having a central bore therethrough, a compression tube positioned within the central bore, an annular elastomeric seal positioned within the central bore distal to the compression tube, and at least one actuator configured as a lever attached to the housing, with a bias (e.g., spring). The at least one actuator may have a distal rotating cam surface that is configured to move in a first direction and a second direction. Movement of the lever in the first direction moves the rotating cam surface against the compression tube and drives the compression tube distally against the elastomeric seal to reduce an inner diameter of the annular elastic seal as the annular elastomeric seal is axially compressed (e.g., against a distal wall or rim of the bore). Movement of the lever in the second direction moves the rotating cam surface against the compression tube and drives the compression tube proximally expanding the annular elastomeric seal axially to increase an inner diameter of the annular elastic seal.

Any of these hemostasis valves can include one or more of the following features. The spring can include a torsion spring, a compression spring, a tensile spring, a leaf spring, or an elastomeric element. The elastomeric seal can be adjacent to the compression tube. The elastomeric seal can include a polymer (e.g., a silicone) with a Shore 00 hardness of between about Shore 00-05 to 00-60 (e.g., about Shore 00-20 to Shore 00-40). The at least one actuator may be at least one lever connected to the housing with a pivot pin. The housing can be connected to a rigidizing catheter. The housing can further include a pressure or vacuum port configured to provide an inlet for pressure of vacuum to rigidize the rigidizing catheter. The valve can further include a flush port. The housing can further include a locking mechanism configured to mate with a locking mechanism of a device passing through the central bore. The spring can bias the lever in the first direction.

In some examples the hemostasis valve includes a cylindrical tube and an annular collar. The cylindrical tube has a constant outer diameter and a tapered inner surface surrounding a central bore. The annular collar may be positioned around the cylindrical tube and configured to move axially relative to the cylindrical tube to seal a device within the central bore. The cylindrical tube may further include a plurality of axially extending flanges.

In general, a number of the features described herein have been found to be surprisingly effective in preventing leakage, particularly in larger-diameter hemostasis valves as described herein; for example, the hemostasis valves described herein (which may be referred to as “large-bore hemostasis valves” may be configured to fully seal and to open to allow passage of a medical device having a diameter of up to 12 mm (e.g., greater than 36 French). This is due, in part, to the softness of annular elastomeric seal. The annular elastomeric seal may be formed of a very soft material, such as a material have a Shore 00 hardness of between about Shore 00-05 and about Shore 00-60 (e.g., Shore 00-10 and Shore 00-40, e.g., Shore 00-15 and Shore 00-35, e.g., less than Shore 00-80, less than Shore 00-70, less than Shore 00-60, etc.). These highly soft annular elastomeric seal may function far better than more traditional “soft” materials. Outside of the Shore 00 hardness ranges described above, the valve may leak, and may require very high levels of force (pressure) to close.

In addition, any of these apparatuses may include a lubricious material on or around the soft annular elastomeric seal. The annular elastomeric seal may generally be positioned within a region of the central bore of the apparatus and may be compressed by the slideable compression tube. In operation, the annular elastomeric seal may be compressed or allowed to relax within this region of the central bore of the apparatus. If a lubricious material (e.g., parylene) is included on the annular elastomeric seal and/or within this region of the central bore of the apparatus it may prevent the annular elastomeric seal from sticking to itself and/or to the walls of the central bore and/or the compression tube. The lubricious material on the inner surface of the annular elastomeric seal helps for the lower-force passage of devices through the central bore. The use of a lubricant in this manner has surprisingly been found to dramatically reduce leakage, which may be due to sticking of the annular elastomeric seal.

In contrast to hemostasis valves that require the use of a compression spring in-line with the sealing element (e.g., the annular elastomeric seal), which may require higher forces to actuate (e.g., open) and/or may result in leaking, the apparatuses described herein may instead use one or more actuators to control the position of a compression tube and therefore the configuration of the annular elastomeric seal. The actuator may be biased in the relaxed (unconstrained state) to compress the annular elastomeric seal via a bias, such as a spring, coupling the actuator to the housing, rather than acting directly on the compression tube, or by acting through an intermediate element that is in-line with that central bore. This configuration may allow sealing even against high pressures without requiring large forces to open the valve by actuating the one or more actuator. The attachment of the bias between the actuator and the housing may also provide a more advantageous assembly. It may also serve to reduce the minimal radial diameter of the unit.

For example, described herein are hemostasis valves, comprising: a housing having a central bore therethrough; a compression tube movably positioned within the central bore; an annular elastomeric seal within the central bore adjacent to the compression tube; and at least one actuator movably coupled to the housing, the at least one actuator having one or more engagement surfaces engaging with one or more driving surfaces on the compression tube, the at least one actuator configured to move in a first direction and a second direction; wherein movement of the at least one actuator relative to the housing in the first direction moves the compression tube to compress the annular elastomeric seal to reduce an inner diameter of the annular elastomeric seal, and wherein movement of the at least one actuator relative to the housing in the second direction moves the compression tube to provide for axial expansion the annular elastomeric seal to increase an inner diameter of the annular elastomeric seal; and a bias coupled to the at least one actuator and configured to urge the at least one actuator in the first direction when the hemostasis valve is in a neutral state.

In any of these apparatuses, the compression tube may be coupled (e.g., bonded), either directly or indirectly to the annular elastomeric seal. The compression tube may be rigidly coupled to the annular elastomeric seal, so that axial movement of the compression tube (e.g., proximally-to-distally/distally-to-proximally) pulls and pushes the annular elastomeric seal. The compression tube may be bonded to the annular elastomeric seal on a first side of the annular elastomeric seal. The compression tube may be bonded by an adhesive material.

In any of these apparatuses the compression tube may not require a compression spring in-line with the compression tube to bias the compression tube to compress the annulare elastomeric seal. As described above, instead, the annular elastomeric seal may be held in the closed (sealed) configuration at rest by a bias or biases between the actuator(s) and the housing.

Any of these apparatuses, e.g., hemostasis valves and/or systems including them, may include a lubricous material within the region of the central bore in communication with the annular elastomeric seal, so that the annular elastomeric seal may slide relative to the central bore. For example, the lubricious material may be applied (coated, etc.) to the annular elastomeric seal, and/or may be applied to the region of the central bore housing or holding the annular elastomeric seal. The lubricious material may be a fluid, gel, powder, etc. In some examples the lubricious material may be parylene.

The actuators may be, e.g., a lever, dial, knob, etc. In some examples the actuator comprises a lever. In some examples the actuator comprises a dial. For example, the actuator may comprise a threaded knob that may be rotated clockwise or counterclockwise to modify the position of the compression tube and therefore the configuration of the annular elastomeric seal.

The one or more engagement surfaces may comprise one or more of: a cam, a gear or rack, a threaded region, a linkage. For example the one or more engagement surfaces may comprise a cam engaging a driving surface on the compression tube.

As mentioned, any of these apparatuses may include a bias driving the at least one actuator in the first direction, e.g., so that the device is sealed closed at rest. The bias may be coupled between the actuator and the housing. For example, the bias may be a torsion spring coupling the at least one actuator to the housing.

As mentioned, the annular elastomeric seal may be very soft; for example the annular elastomeric seal may have a Shore 00 hardness of between Shore 00-05 and 00-60. The elastomeric seal may comprise a material such as a silicone material or other polymeric material. In some examples the elastomeric seal has a Shore 00 hardness of between Shore 00-10 and 00-40.

The at least one actuator may be pivotally coupled to the housing with a pivot pin. The pivot pin might be a separate component, or it could be a co-joined feature with either the housing or the at least one actuator.

Any of the apparatuses described herein may include a rigidizing catheter coupled to the housing. Examples of rigidizing catheters may include those described in U.S. patent application Ser. No. 17/152,706, titled “DYNAMICALLY RIGIDIZING COMPOSITE MEDICAL STRUCTURES”, now U.S. patent application Ser. No. 11,135,398, herein incorporated by reference in its entirety. Thus, any of these hemostatic valves described herein may include a port configured as an inlet for the application of pressure of vacuum to rigidize the rigidizing catheter, and/or a flush port.

Any of these hemostasis valves may include a locking mechanism on the housing configured to mate with a locking mechanism of a device passing through the central bore. In some examples the apparatus may include a handle retainer configured to secure the one or more lever arms so that the compression tube does not substantially compress the elastomeric seal so that the inner diameter of the annular elastomeric seal is open to form a channel therethrough. In some examples the compression tube is coupled to (e.g., bonded to) the annular elastomeric seal so that when the actuator (e.g., handle) is moved in the second direction to open the lumen through the annular elastomeric seal, or is held in the second position to hold the annular elastomeric seal open, the annular elastomeric seal may be pulled axially and/or allowed to axially expand, e.g., self-expand) so that the lumen through the annular elastomeric seal is opened.

For example, a hemostasis valve may include: a housing having a central bore therethrough; a compression tube movably positioned within the central bore; an annular elastomeric seal within the central bore adjacent to the compression tube, wherein the annular elastomeric seal has a Shore 00 hardness of between 00-05 and 00-60; a lubricous material on annular elastomeric seal configured to prevent the elastomeric seal from sticking; and at least one actuator movably coupled to the housing, the at least one actuator (e.g., at least one lever) having one or more engagement surfaces engaging with one or more driving surfaces on the compression tube, the at least one actuator configured to move in a first direction and a second direction; wherein movement of the at least one actuator relative to the housing in the first direction moves the compression tube to compress the annular elastomeric seal to reduce an inner diameter of the annular elastomeric seal, and wherein movement of the at least one actuator relative to the housing in the second direction moves the compression tube to pull the annular elastomeric seal so that it expands axially to increase an inner diameter of the annular elastomeric seal.

A hemostasis valve may include: a housing having a central bore therethrough; a compression tube movably positioned within the central bore; an annular elastomeric seal within the central bore adjacent to the compression tube, wherein the annular elastomeric seal has a Shore 00 hardness of between 00-05 and 00-60; a lubricous material on annular elastomeric seal configured to prevent the elastomeric seal from sticking; at least one actuator movably coupled to the housing, the at least one actuator having one or more engagement surfaces engaging with one or more driving surfaces on the compression tube, the at least one actuator configured to move in a first direction and a second direction; wherein movement of the at least one actuator relative to the housing in the first direction moves the compression tube towards the elastomeric seal to reduce an inner diameter of the annular elastomeric seal, and wherein movement of the at least one actuator relative to the housing in the second direction moves the compression tube to allow the elastomeric seal to expand laterally (and/or to pull the annular elastomeric seal) so that the annular elastomeric seal increases it's lumen diameter (opening the valve). The apparatus may also include a bias driving the at least one actuator in the first direction when the hemostatic valve is in the neutral state, so that the hemostatic valve is closed.

All of the methods and apparatuses described herein, in any combination, are herein contemplated and can be used to achieve the benefits as described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1C show an exemplary hemostasis valve. FIG. 1A shows a cross section of the valve in an open unsealed position. FIG. 1B shows an isometric view of the valve of FIG. 1A. FIG. 1C shows a cross section of the valve in a closed sealed position.

FIGS. 2A-2D show an exemplary hemostasis valve with a device passing therethrough. FIG. 2A shows a cross section of the valve in a partially open unsealed position with the device passed partially therethrough. FIG. 2B shows a side view of the valve and device of FIG. 2A. FIG. 2C shows a cross section of the valve in a sealed position around the fully inserted device. FIG. 2D shows an isometric view of the valve and device of FIG. 2C.

FIG. 3 shows an exemplary hemostasis valve with a clip therearound.

FIGS. 4A-4B show an exemplary hemostasis valve in packaging.

FIGS. 5A-5D show another exemplary hemostasis valve. FIG. 5A shows an end view. FIG. 5B shows a cross-section. FIG. 5C shows a side view. FIG. 5D shows a device passed through the valve.

FIGS. 6A-6C illustrate an example of a hemostasis valve similar to that shown in FIGS. 1A-1C including an actuator configured as a lever having a camming engagement surface engaging driving surfaces on the compression tube. FIG. 6A shows a perspective view. FIGS. 6B and 6C show a top view and a section through a top view, respectively.

FIGS. 7A-7C illustrate an example of a hemostasis valve including an actuator configured as a lever having a geared engagement surface engaging a driving surfaces on the compression tube. FIG. 7A is a perspective view. FIGS. 7B and 7C show a top view and a section view, respectively.

FIGS. 8A-8C illustrate an example of a hemostasis valve including an actuator configured as a rotatable knob having threaded engagement surface engaging the compression tube. FIG. 8A shows a perspective view. FIGS. 7B and 8C show a top view and a section view, respectively.

FIGS. 9A-9C illustrate an example of a hemostasis valve including an actuator configured as a linkage engaging the compression tube. FIG. 9A shows a perspective view. FIGS. 9B and 9C show a top view and a sectional view, respectively.

FIGS. 10A-10B illustrate one example of a handle retainer configured to engage a hemostasis valve and secure the actuator so that the valve is maintained in the open configuration.

FIGS. 10C-10D illustrate the handle retainer of FIGS. 10A-10B with a hemostasis valve secured within.

### DETAILED DESCRIPTION

Described herein are hemostasis valves. Advantageously, the hemostasis valves described herein provide a consistent seal when devices with a range of diameters are extended therethrough. For example, the hemostasis valves described herein are effective with 12, 16, 20, 24, 30, and 36 French devices, so that the inner diameter of the seal may convert from fully and securely sealed to up to 12 mm or larger in inner diameter. The hemostasis valves described herein are advantageously leak-free and stable at high hemostatic pressures, including pressures as high as 38 kPa (5.5 Psi). Additionally, the hemostasis valves described herein advantageously enable devices passed therethrough to slide with low drag. The hemostasis valves described herein can be dynamically adjustable so as to adjust the drag to substantially zero by adjusting the one or more actuators. Finally, the hemostasis valves described herein are easy to operate, requiring low force and only a single hand (e.g., only two fingers from a single hand).

The advantages described above may be attributed, at least in part, due to the configuration and arrangement of the soft annular elastomeric seal, and the engagement of the actuator(s) with the housing and with the compression tube movably positioned within the central bore of the housing, as well as the return spring location and configuration. For example, the soft annular elastomeric seal may be formed of a very soft material, e.g., having a Shore 00 durometer of between 00-05 and 00-60 (e.g., between 00-20 and 00-040, etc.), and may be lubricated and/or enclosed in a lubricious material to allow it to move (contract and expand) within the central bore of the housing without sticking to itself or to the housing. The actuator may be one of a number of different actuators descried herein that may be adjustable from outside of the housing and that engage with the compression tube via one or more engagement surfaces engaging with one or more driving surfaces on (or rigidly coupled to) the compression tube. A bias (e.g., spring) may be coupled between the actuator(s) and the housing to maintain the compression tube in a position so that it compresses the annular elastomeric seal when the apparatus is at rest with force sufficiently high to maintain the seal and prevent leakage, but low enough to allow relatively easy manual operation of the actuator to open the valve by moving the compression tube away from the annular elastomeric seal, pulling or allowing the annular elastomeric seal to laterally expand and open or increase the lumen through the annular elastomeric seal.

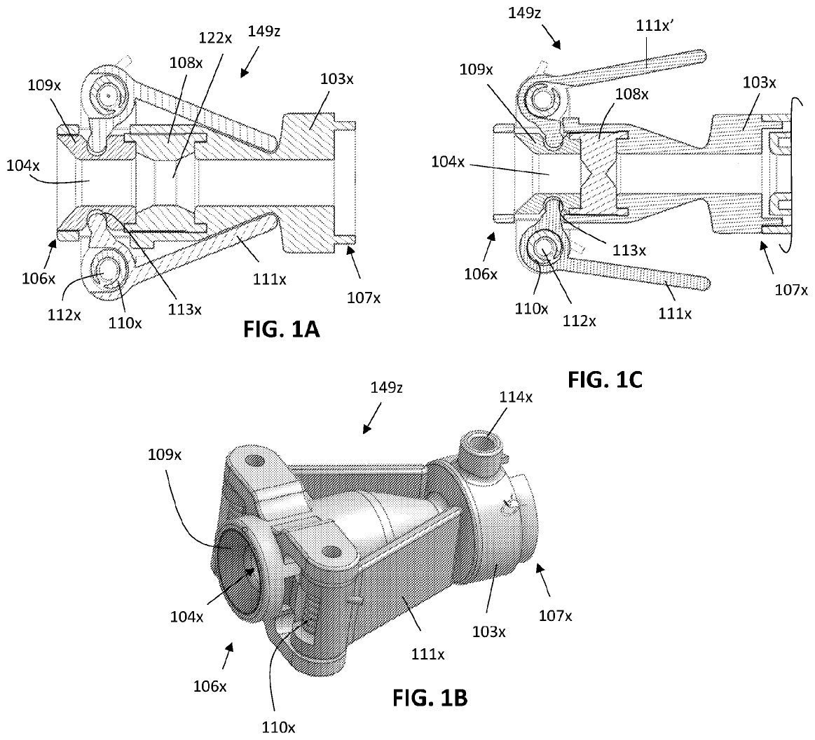

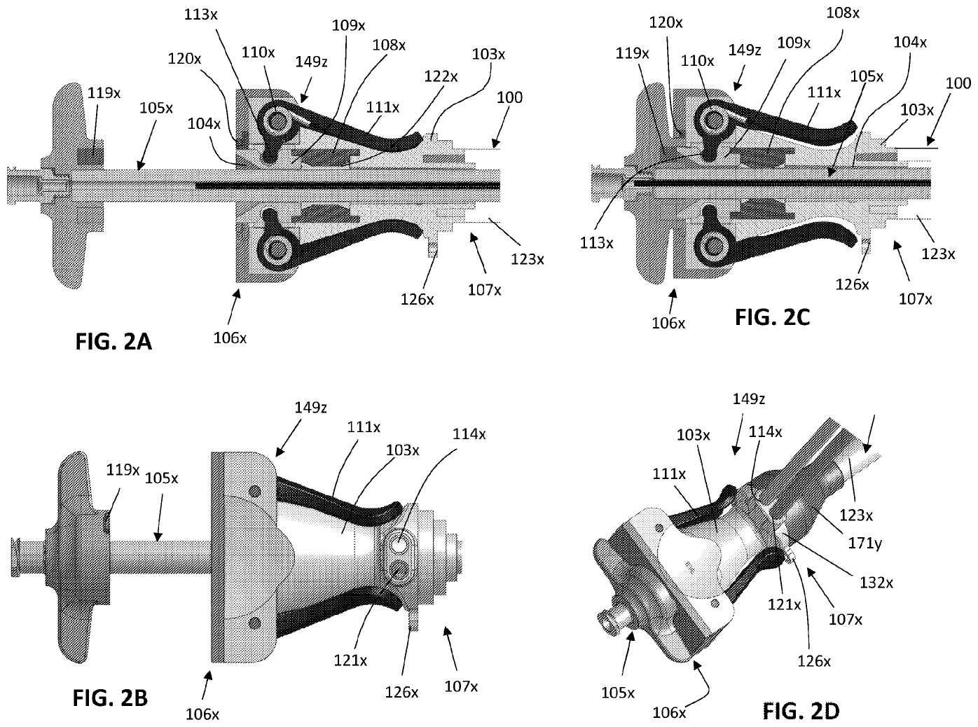

FIGS. 1A-1C illustrate an example of a hemostasis valve 149z including a housing 103x with a central bore 104x configured to enable a device 105x (see FIGS. 2A-2D), such as a catheter or dilator, to pass therethrough. As shown, the central bore 104x can house a substantially cylindrical (or annular) elastomeric seal 108x and a compression tube 109x therein. The compression tube 109x can be positioned within the central bore 104x adjacent (either directly adjacent or indirectly adjacent) to the annular elastomeric seal, e.g., at the proximal end 106x of the valve 149z and can have a funneled inlet to enable easier passage of a device 105x therethrough. The elastomeric seal 108x can be adjacent to (and/or attached to) the compression tube 109x and can be bordered at the distal end by the housing 103x. In some examples, the elastomeric seal 108x can be made of a very soft silicone, having a Shore 00 durometer of between about Shore 00-05 and Shore 00-60. Two actuators 111x, configured a levers in FIGS. 1A-1C, are movably coupled to the housing 109x via a pin pivot 112x; each actuator is also biased in a particular position (at rest) by a bias (e.g., torsion spring) 110x driving the actuator relative to the housing 103x. Each actuator 111x also includes engagement surfaces configured as rotating cam surfaces 113x. The pivot pin 112x may be formed as part of the housing or may be a separate element.

The distal end 107x of any of the hemostasis valves described herein can be connected to a catheter 100, such as a dynamically rigidizing catheter. Exemplary dynamically rigidizing catheters are described in International Application Nos. PCT/US2018/042946, filed on Jul. 19, 2018, titled “DYNAMICALLY RIGIDIZING OVERTUBE,” PCT/US2019/042650, filed on Jul. 18, 2019, titled “DYNAMICALLY RIGIDIZING COMPOSITE MEDICAL STRUCTURES,” and PCT/US2020/013937, filed on Jan. 16, 2020, titled “DYNAMICALLY RIGIDIZING COMPOSITE MEDICAL STRUCTURES,” the entireties of which are incorporated by reference herein. FIGS. 2A-2D (and FIG. 1C) illustrates a hemostasis valve such with a catheter attached. The catheter 100 can include a main elongate body 123x (e.g., a rigidizing elongate body) and a proximal hub 171y (shown in FIG. 2D). The proximal hub 171y can be configured to mate with (e.g., permanently attach to) the distal end 107x of the valve 149z. The housing may include a shroud 132x that has a pressure or vacuum port 121x configured to provide an in inlet for pressure or vacuum to rigidize the main elongate body 103x. In some examples, and as shown in FIGS. 2A-2D, the valve 149z can further include a flush port 114x.

As is also shown in FIGS. 2A-2D, a device 105x (e.g., a dilator) can be configured to pass through the central bore 104x, e.g., at the proximal end 106x of the valve 149z, and exit through the distal end 107x of the valve 149z, e.g., into the lumen of a catheter 100 coupled to the hemostasis valve. In some examples, the device 105x can include a mating mechanism 119x (e.g., a bayonet feature) at the proximal end thereof configured to attach to a corresponding mating mechanism 120x at the proximal end 106x of the valve 149z to stabilize the device 105x relative to the valve 149z. In some examples, the valve 149z can further include a tie-off port 126x configured to enable attachment of the valve 149z to the patient.

Referring still to FIGS. 2A-2D, in use of the valve 149z, a device 105x can be placed within the bore 104x. In a neutral position, the torsion springs 110x can bias the levers 111x outwards, and the seal 108x can be closed (thereby sealing the bore 104x similar to as shown in FIG. 1C). As shown in FIGS. 2A-2B, to enable insertion of the device 105x through the bore 104x, the levers 111x can be actuated (i.e., pushed inwards) by a user (e.g., each lever 111x can be actuated with a single finger, and both levers 111x can be actuated simultaneously with a single hand). As the levers 111x are closed, the rotating cam surface 113x can push proximally against the compression tube 109x, causing the compression tube 109x to move proximally relative to the housing 103x, thereby releasing compression on the elastomeric seal 108x. As the elastomeric seal 108x is uncompressed, it can elongate axially, and the wall of the seal 108x can reduce in thickness (i.e., such that the diameter of the central lumen 122x of the seal 108x expands). With the central lumen 122x expanded, the device 105x can slide through the bore 104x and into the lumen of the catheter 100. As shown in FIGS. 2C-2D, when the device 105x is in the desired position (e.g., with the mating mechanisms 119x/120x attached), the actuators 111x can be reversed (i.e., released so as to move outwards). As a result, the engagement surface (e.g., cam surface 113x) can push distally against a driving surface of the compression tube 109x, causing the compression tube 109x to move toward the annular elastomeric seal (in this example, moving the compression tube distally). Distal movement of the compression tube 109x towards the elastomeric seal correspondingly compresses the elastomeric seal 108x. The elastomeric seal is radially and axially constrained within the central bore of the housing, thus driving the compression tube against the elastomeric seal forces the seal 108x to compress against the outer housing 103x such that the central lumen 122x through the annular elastomeric seal is reduced. The reduction of the lumen 122x through the annular elastomeric seal can enable complete sealing around the device 105x. This is illustrated by comparison of FIG. 1A, showing the elastomeric seal 108x in an uncompressed configuration with the lumen 122x open, and FIG. 1B, showing the elastomeric seal 108x compressed by the compression tube 109x so that the lumen 122x is closed.

The amount of sealing achieved by the hemostasis valve 149z can advantageously be dynamically adjusted during use (e.g., during a procedure with the device 105x) by adjusting the position of the actuators 111x. For example, the user may feel the tactile feedback when operating the actuator indicating the compression of the soft annular elastomeric seal, and may adjust the seal, e.g., to reduce or increase drag on a medical device within the hemostatic valve as desired.

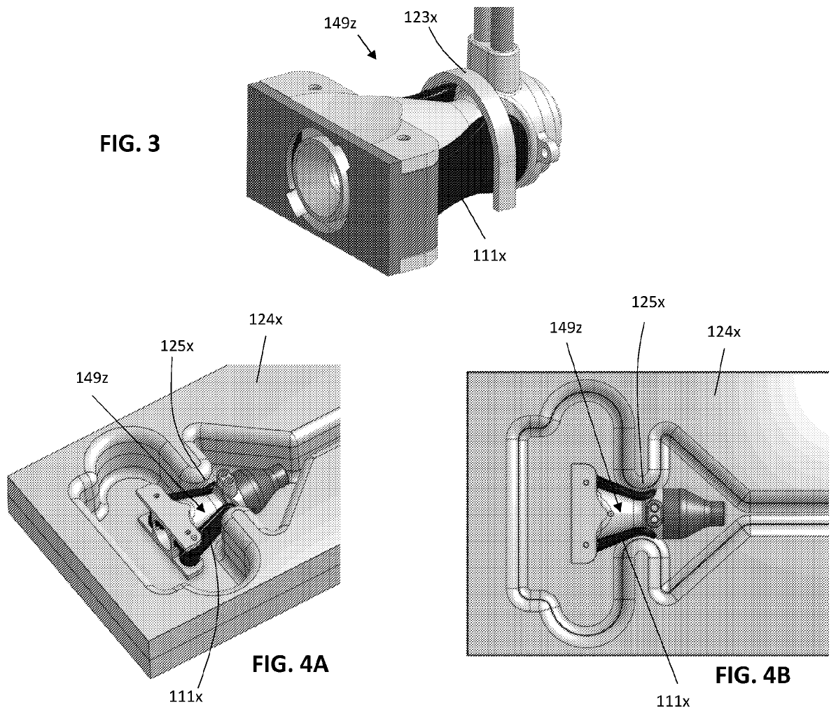

Any of the hemostatic valves described herein may include a retainer or clip (e.g. handle retainer) to secure the position of the valve (e.g., open, closed partially open/closed) by securing the actuator. For example, FIG. 3 includes a retainer 123x, shown as a lever compression clip, configured to keep the actuators (levers) 111x closed and the elastomeric seal 108x un-stressed (and open) during storage and/or before use. The clip 123x can be, for example, removed and disposed of before product use.

Referring to FIGS. 4A and 4B, in some examples, the packaging 124x for the system (e.g., including the valve device 149z and the catheter) can be configured to hold the actuator(s) 111x so that the valve is maintained open. For example, the packaging 124x can be a thermoformed tray having constraints 125x configured to engage with and maintain the levers 111x in the closed position. As the system is removed from the tray, the constraints 125x can become disengaged from the actuators (e.g., levers) 111x, allowing the actuators 111x to open. Similarly, in some examples, the packaging can include a strap or clip that keeps the actuators 111x closed. In this example, the strap or clip can remain with the packaging when the valve 149z is removed from the packing for use.

In some examples, a vacuum source (for example, a syringe or a vacuum pump) can be attached and/or sealed to the hemostatic valve, e.g., the proximal end 106x of the valve 149z, to enable removal of blood and/or blood clots via vacuum. In any of the apparatuses described herein, the length of the hemostatic valve and/or of the component parts of the lumen of the hemostatic valve may be relatively short to reduce the “dead volume” within the valve. For example, the distance between the annular elastomeric seal and the distal end of the device may be 8 cm or less (e.g., 7 cm or less, 6 cm or less, 5 cm or less, 4 cm or less, 3 cm or less, etc.).

In some examples, the bias that biases the actuators relative to the housing may be torsions springs 110x as shown in FIGS. 1A-1C and 2A-2D. In some examples the bias can be one or more leaf springs (e.g., under each actuator 111x), one or more compression springs (e.g., along the central bore 104x, under each actuator 111x, or between the actuators 111x across the top of the housing at the proximal end 106x), or one or more tension springs. In some examples, the bias (e.g., torsion spring, leaf spring, compression spring, or tension springs) can be made of an elastomer, such as an elastomeric cylinder or band.

Any appropriate actuator or combination of actuators may be used. FIGS. 1A-1C and 2A-2D illustrate actuators configured as levers 111x. However, in some examples the actuators can be, e.g., rotating knobs or dials, linkages, and/or buttons that move, for example, orthogonal to the bore 104x.

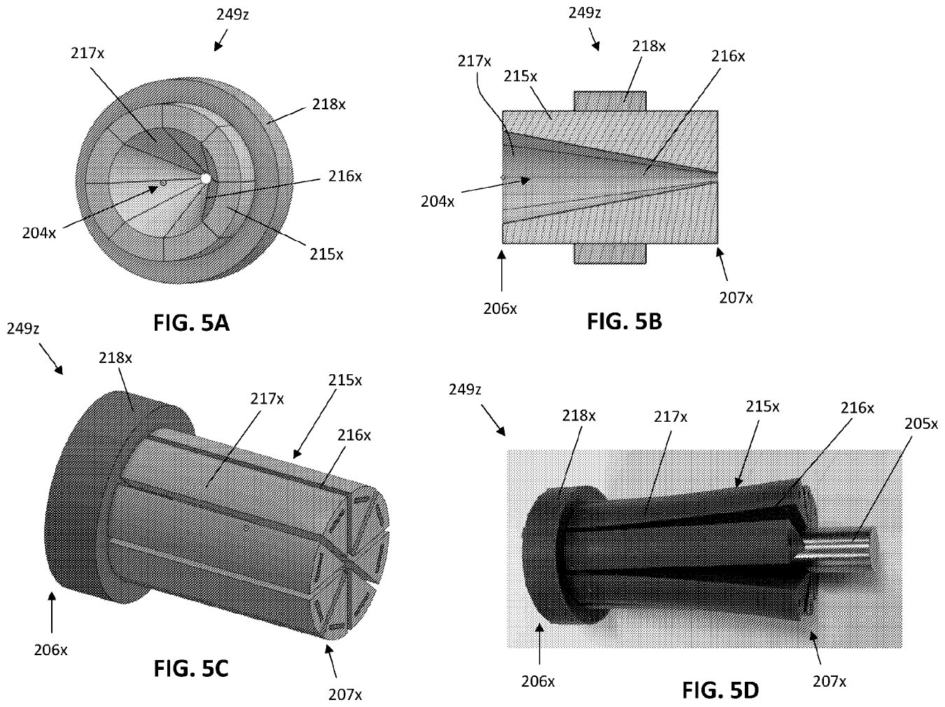

Another exemplary example of a hemostasis valve 249z is shown in FIGS. 5A-5D. The hemostasis valve 249z in this example includes a cylindrical tube 215x (e.g., made of an elastomer) surrounding a central bore 204x. The cylindrical tube 215x can be include a constant outer diameter and a tapered inner surface (along the bore 204x). The tapered inner surface can have a diameter that is larger at the proximal end 206x and shrinks to zero at the distal end 207x. Further, the cylindrical tube 215x can include a plurality of distal flanges 217x at the distal end 207x of the valve 249z. The distal flanges 217x can be separated by axial slits 216x extending from the distal end 207x of the valve 249z towards the proximal end 206x of the valve 249z. The flanges 217x can advantageously enable the cylindrical tube 215x to radially expand as a device 205x is passed through the central bore 204x (i.e., the flanges 217x can move radially outwards and the slits 216x widen, as shown in FIG. 5D). The valve 249z can further include an annular collar 218x positioned around the cylindrical tube 215x. The annular collar 218x can be configured to move axially along the outer surface of the cylindrical tube 215x.

In use of the valve 249z, a device 205x can be positioned through the bore 204x. As the device 205x is moved from the proximal end 206x to the distal end 207x, the device 205x can push the flanges 217x radially outwards. Moreover, the annular collar 218x can be moved distally by the user (e.g., with a single hand) along the outer diameter of the tube 215x until the desired seal is created between the flanges 217x and the device 205x. In some examples, the annular collar 218x can be moved axially (proximally or distally) during use (i.e., during a procedure with the device 205x) to dynamically adjust the desired amount of seal and drag with the device 205x.

In some examples, the valve 249z can include a spring configured to push the annular collar 218x in the tight (i.e., in the distal) direction, thereby applying a predetermined load to ensure complete hemostatic sealing. To introduce the device 205x into the bore and/or to temporarily reduce the seal (and thus to reduce the drag), the user can push the annular collar 218x against the spring in the proximal direction. When the user releases the annular collar 218x, the spring can move the annular collar 218x distally again to ensure hemostatic sealing.

Advantageously, the hemostasis valves 149z, 249z described herein can function with a wide range of device diameters, such as up to and above 24Fr. Additionally, the valves 149z, 249z can provide sealing across a full continuum of device diameters and not just discrete sizes. The valves 149z, 249z can be adjusted so as to reduce the amount of sealing/drag and, conversely, so as to increase the amount of sealing/drag when desired. The valves 149z, 249z are advantageously simple to operate (e.g., with only two fingers and a single hand) and do not require additional accessories (e.g., do not require a syringe).

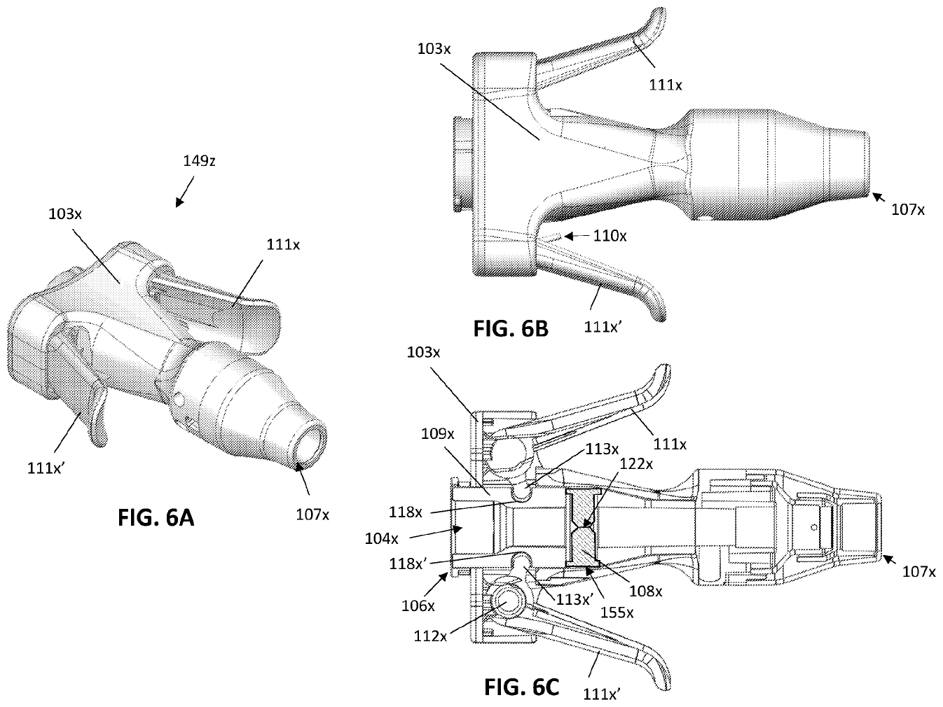

FIG. 6A-6C illustrate another example of a hemostatic valve similar to that shown in FIGS. 1A-1C. In this example, the valve 149x includes a housing 103x with a central bore 104x. A pair of actuators 111x, 111x′ are pivotally linked to the housing. The actuators are shown as levers in this example and may be coupled to the housing by a pin 112x which may be formed as part of the housing. Alternatively the pin may be formed as part of the actuator and may engage a socket in the housing. The end 107x of the apparatus may be configured to mate with a catheter, such as a rigidizing catheter, as described above. A bias 110x (shown in this example as a torsional spring in FIG. 6B) is included at each actuator to bias the actuator relative to the housing. For example, as shown in FIG. 6C the bias maintains the actuator (levers 111x, 111x′) in an open configuration away from the housing so that the engagement surfaces 113x, 113x′ of each actuator, in this example shown as cam surfaces, engage with driving surfaces 118, 118′ of the compression tube 109x to drive the compression tube towards the annular elastomeric seal 108x so that it is compressed within the region of the bore 104x through the housing to close the central lumen 122x through the annular elastomeric seal 108x. Moving the actuators 111x, 111x′ relative to the housing by pivoting them in a second direction (opposite from the first direction they are biased in at rest) drives the engagement surface against the driving surface of the compression tube, moving the compression tube toward the proximal end 106x of the apparatus, and so that the annular elastomeric seal expands laterally (and in some cases is pulled laterally) to contract radially, opening the annular elastomeric seal. As mentioned above, either or both the annular elastomeric seal or the region 155x of the central bore of the housing in which the annular elastomeric seal 108x resides may be lubricated and/or may include a lubricious material (solid, liquid, gel, etc. such as a coating). In some cases the solid, very low durometer materials used to form the annular elastomeric seal may be somewhat sticky or tacky, which may negatively impact their ability to expand and contract smoothly and predictably in operation. Thus, the use of a lubricous material in conjunction with the annular elastomeric seal may dramatically improve the performance of the hemostatic valve.

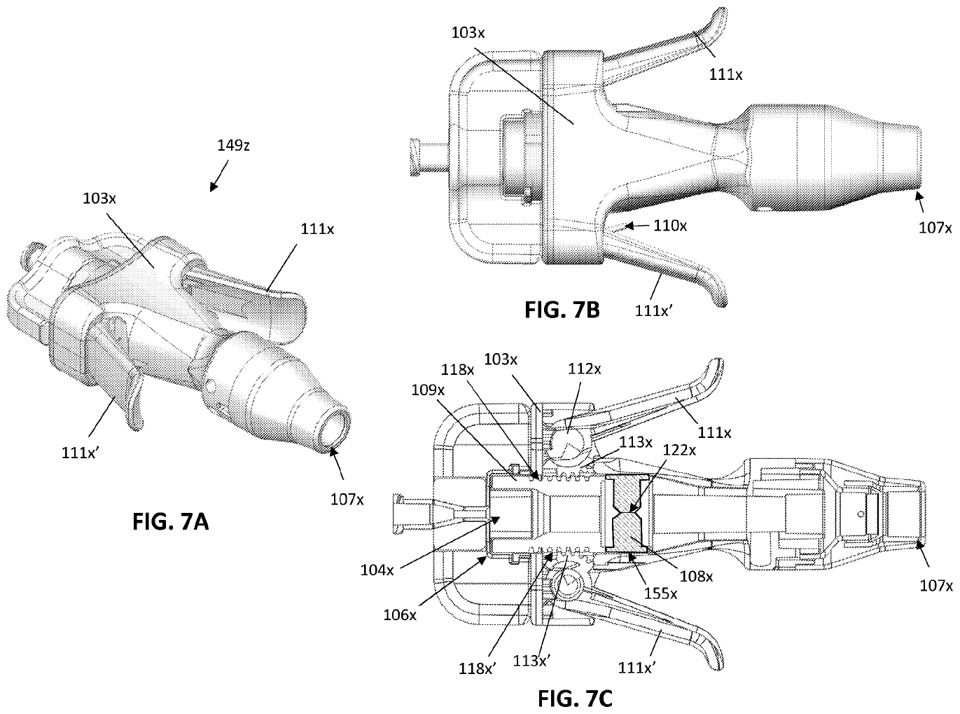

FIGS. 7A-7C show another example of a hemostatic valve 149z in which the actuators 111x, 111x′ are also configured as levers that are pivotally connected to the housing 103x and biased by a spring 110x, however in this example the engagement surface 113x of each actuator is configured as a gear. As shown in FIG. 7C, each actuator 111x is rigidly coupled (or integral with) a toothed gear 113x forming the engagement surface; the teeth of the engagement surfaces engage with a complimentary toothed driving surface 118x that is formed on (or rigidly coupled to) the compression tube 109x. For example the outer diameter of the compression tube may include ridges or channels forming the driving surface 118x, 118x. Thus, movement of the actuators 111x, 111x′ as they pivot 112x relative to the housing 103x may drive the compression tube towards or away from the annular elastomeric seal 108x to compress or expand laterally (and correspondingly to expand radially inward or retract radially outward, respectively) the annular elastomeric seal. In FIG. 7C, the actuators 111x, 111x′ are biased outward so that the toothed engagement surfaces 113x lock the compression tube 109x in compression against the annular elastomeric seal 108x so that the seal is closed (e.g., closing the lumen 122x through the annular elastomeric seal). In general, any of the hemostasis valves described herein may include a lock or locks securing the actuator in a particular position so that the compression tube is also locked in a predefined position. In general, locking the actuators in position may lock the annular elastomeric seal (and the compression tube).

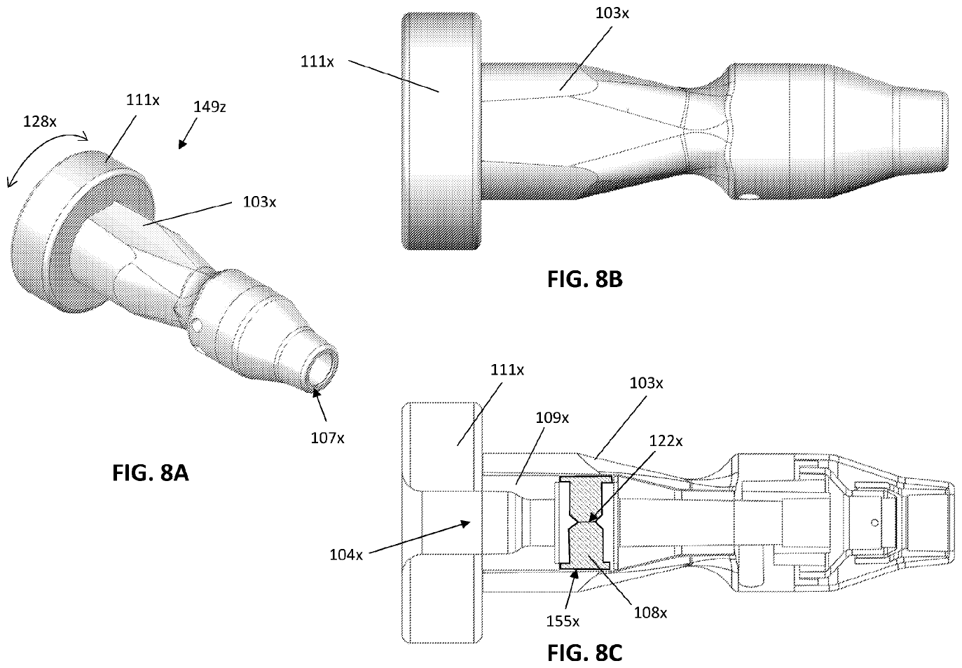

FIGS. 8A-8C illustrate another example of a hemostatic valve 149z in which the actuator 111x is configured as a rotatable knob or dial that may rotate 128x relative to the housing 103x. The actuator may also be biased (e.g., by a spring). Rotating the actuator relative to the housing 103x may allow a threaded connection between an engagement surface of actuator (not shown) and a driving surface of the compression tube 109x to move relative to each other, advancing, retracting, and/or twisting the compression tube within the central bore 104x. For example the compression tube may include helical threads that are engaged with a projection or projections forming the engagement surface(s) of the actuator. Rotating the actuator 111x, which may or may not be in a longitudinally fixed position relative to the housing 103x may therefore change the relative position of the compression tube with respect to the housing 103x. In some examples, rotation of the actuator 111x relative to the housing 103x may therefore drive the compression tube towards or away from the annular elastomeric seal 108x to compress or expand laterally (and correspondingly to expand radially inward or retract radially outward, respectively). Alternatively or additionally, in some examples rotation of the actuator 111x may drive rotation of the compression tube 109x. The compression tube may be rigidly coupled to the annular elastomeric seal 108x so that rotation of the compression tube causes rotation of one axial end of the annular elastomeric seal, twisting the annular elastomeric seal. The end of the annular elastomeric seal in any of the example apparatuses described herein may be coupled (e.g., bonded) to the central bore of the housing. This torsional twisting of the annular elastomeric seal may result closing of the lumen through the annular elastomeric seal, closing the seal. In some examples the compression tube may be actuated by the actuator to both rotate and to axially compress/expand.

In FIG. 8C the annular elastomeric seal 108x is shown in a compressed configuration with the lumen 112x through the seal closed. Alternatively, the elastomeric seal may close due to torsion, which may or may not require any axial advancement.

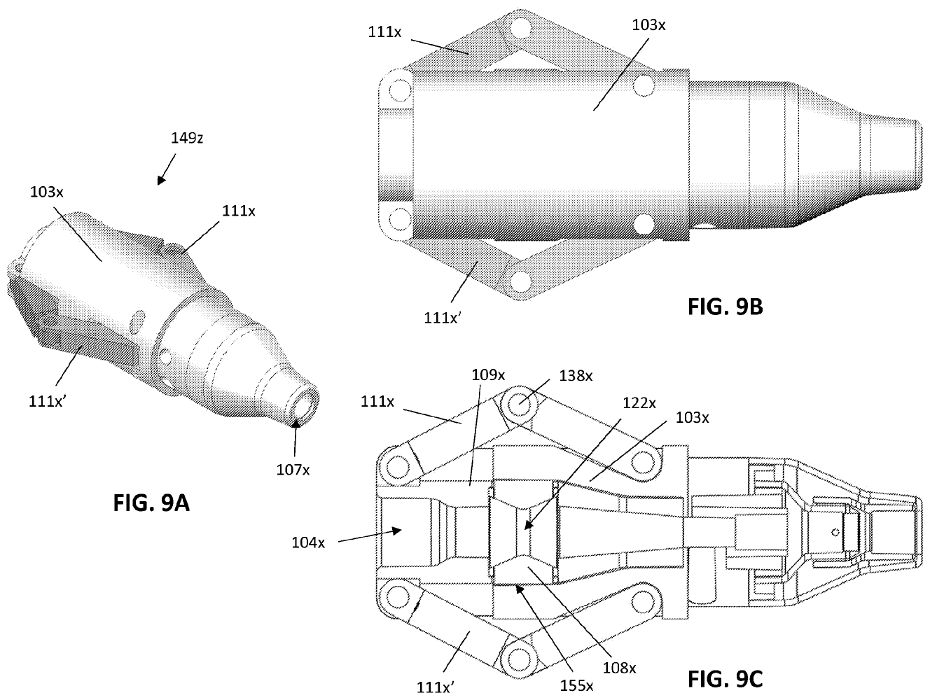

FIGS. 9A-9C illustrates an example of a hemostatic valve 149z having a pair of actuators 111x, 111x′ that are configured as linkages that control the position of the compression tube 109x relative to the housing 103x. As shown in FIG. 9C, each actuator includes a pair of linkage arms that are pivotally connected to each other and to the housing 103 at one end and to the compression tube 109x on the other end. The compression tube may be slid within the central bore (passage) 104x of the housing towards or away from the annular elastomeric seal 108x, in order to apply or remove compression causing the annular elastomeric seal 108x to expand radially inward to close the lumen 122x or to withdraw radially outward to open the lumen 122x. As in any of the examples described herein, the region 155x of the central bore in which the annular elastomeric seal 108x resides, or the annular elastomeric seal itself may be lubricious or may include a lubricant. In some examples, this configuration could provide recoil forces by utilizing one or more compression springs positioned orthogonal to the central axis, e.g., pushing out on the linkages (e.g., at pivot joint 138x, pushing from the housing 103x)

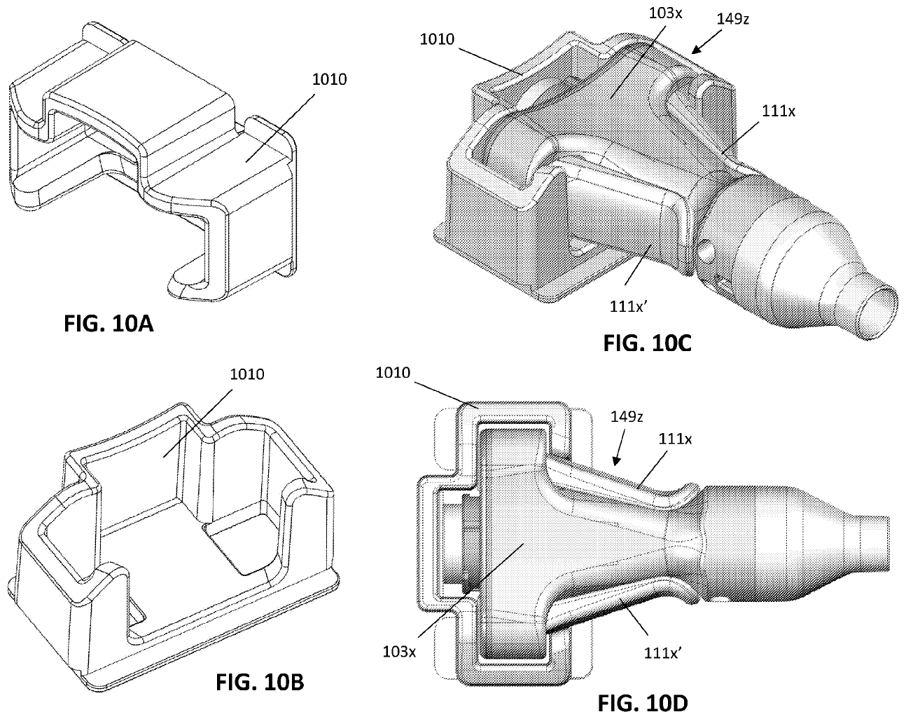

FIGS. 10A-10D illustrate a handle retainer 1010 configured to secure actuators 111x of a hemostasis valve 149z in a selected position so that the compression tube of the hemostasis valve is secured in position relative to the annular elastomeric seal, locking the seal in a predetermined position (e.g., open, closed, partially open, etc.). In FIG. 10B, the handle retainer 1010 is configured to secure the lever arms forming the actuators 111x, 111x′ in a rotated position so that they are held against the housing 103x; in this position the compression tube is maintained away from the annular elastomeric seal so that the inner diameter of the annular elastomeric seal is open to form a channel therethrough.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A large-bore hemostasis valve, the hemostasis valve comprising:
a housing having a central bore therethrough;
a compression tube movably positioned within the central bore;
an annular elastomeric seal within the central bore rigidly coupled to the compression tube wherein the elastomeric seal has a Shore 00 durometer of between 00-05 and 00-60; and
at least one actuator movably coupled to the housing, the at least one actuator having one or more engagement surfaces engaging with one or more driving surfaces on the compression tube, the at least one actuator configured to move in a first direction and a second direction;
wherein movement of the at least one actuator relative to the housing in the first direction moves the compression tube to compress the annular elastomeric seal to reduce an inner diameter of the annular elastomeric seal, and
wherein movement of the at least one actuator relative to the housing in the second direction is configured to pull the annular elastomeric seal so to move the compression tube to provide for axial expansion the annular elastomeric seal to increase an inner diameter of the annular elastomeric seal.

2. The hemostasis valve of claim 1, wherein the compression tube is not biased against the annular elastomeric seal by a compression spring in-line with the compression tube.

3. The hemostasis valve of claim 1, further comprising a lubricous material within a region of the central bore in communication with the annular elastomeric seal, so that the annular elastomeric seal may slide relative to the central bore.

4. (canceled)

5. The hemostasis valve of claim 1, wherein the at least one actuator comprises a lever.

6. The hemostasis valve of claim 1, wherein the at least one actuator comprises a dial.

7. The hemostasis valve of claim 1, where the one or more engagement surfaces comprises one or more of: a cam, a gear, a threaded region, a linkage.

8. The hemostasis valve of claim 1, where the one or more engagement surfaces comprises a cam engaging a driving surface on the compression tube.

9. The hemostasis valve of claim 24, wherein the bias comprises a torsion spring coupling the at least one actuator to the housing.

10. The hemostasis valve of claim 1, wherein the annular elastomeric seal comprises a silicone material.

11. (canceled)

12. The hemostasis valve of claim 1, wherein the annular elastomeric seal has a Shore 00 hardness of between Shore 00-10 and 00-40.

13. The hemostasis valve of claim 1, wherein the at least one actuator pivotally coupled to the housing with a pivot pin.

14. The hemostasis valve of claim 1, further comprising a rigidizing catheter coupled to the housing.

15. The hemostasis valve of claim 14, further comprising a port configured as an inlet for application of pressure or vacuum to rigidize the rigidizing catheter.

16. The hemostasis valve of claim 1, further comprising a flush port.

17. The hemostasis valve of claim 1, further comprising a locking mechanism on the housing configured to mate with a locking mechanism of a device passing through the central bore.

18. The hemostasis valve of claim 1, further comprising a handle retainer configured to secure the one or more actuators so that the compression tube is secured away from the annular elastomeric seal so that the inner diameter of the annular elastomeric seal is open to form a channel therethrough.

19. A large-bore hemostasis valve, the hemostasis valve comprising:
a housing having a central bore therethrough;
a compression tube movably positioned within the central bore;
an annular elastomeric seal within the central bore rigidly coupled to the compression tube, wherein the annular elastomeric seal has a Shore 00 hardness of between 00-05 and 00-60, further wherein surfaces of both ends of the annular elastomeric seal are orthogonal to the central bore;
a lubricous material on the annular elastomeric seal configured to prevent the annular elastomeric seal from sticking; and
at least one actuator, configured as a lever, movably coupled to the housing, the at least one actuator having one or more engagement surfaces engaging with one or more driving surfaces on the compression tube, the at least one actuator configured to move in a first direction and a second direction;
wherein movement of the at least one actuator relative to the housing in the first direction moves the compression tube to compress the annular elastomeric seal to reduce an inner diameter of the annular elastomeric seal, and
wherein movement of the at least one actuator relative to the housing in the second direction moves the compression tube to pull the annular elastomeric seal so that it expands axially to increase an inner diameter of the annular elastomeric seal.

20. A large bore hemostasis valve, the hemostasis valve comprising:
a housing having a central bore therethrough;
a compression tube movably positioned within the central bore;
an annular elastomeric seal within the central bore rigidly coupled to the compression tube, wherein surfaces of both ends of the annular elastomeric seal are orthogonal to the central bore;
at least one actuator movably coupled to the housing, the at least one actuator having one or more engagement surfaces engaging with one or more driving surfaces on the compression tube, the at least one actuator configured to move in a first direction and a second direction;
wherein movement of the at least one actuator relative to the housing in the first direction moves the compression tube to compress the annular elastomeric seal to reduce an inner diameter of the annular elastomeric seal, and
wherein movement of the at least one actuator relative to the housing in the second direction moves the compression tube to pull the annular elastomeric seal so that it expands axially to increase an inner diameter of the annular elastomeric seal.

21. The hemostasis valve of claim 1, wherein surfaces of both ends of the annular elastomeric seal are orthogonal to the central bore.

22. The hemostasis valve of claim 1, wherein a side of the annular elastomeric rigidly coupled to the compression tube is orthogonal to the central bore.

23. The hemostasis valve of claim 1, wherein the central bore is configured to accommodate a medical device having a diameter of 12 mm or more.

24. The hemostasis valve of claim 1, wherein the central bore is configured to hold a vacuum.

25. The hemostasis valve of claim 1, further comprising a bias coupled to the at least one actuator and configured to urge the at least one actuator in the first direction when the hemostasis valve is in a neutral state.
